(19) United States
(12) Patent Application Publication
(10) Pub. No.: US 2011/0276034 A1
(43) Pub. Date: Nov. 10, 2011

(54) STEERABLE STYLET

(75) Inventors: Crissy Tomarelli, Balmain (AU); David Ogle, Cowan (AU)

(73) Assignee: CATHRX LTD, Homebush Bay (AU)

(21) Appl. No.: 13/143,277

(22) PCT Filed: Jan. 8, 2010
(86) PCT No.: PCT/AU2010/000022

§ 371 Date: Jul. 5, 2011

Related U.S. Application Data
(60) Provisional application No. 61/145,036, filed on Jan. 15, 2009.

(51) Int. Cl. A61M 25/00

(52) U.S. Cl. 604/528

(57) ABSTRACT

A steerable stylet includes an elongate tubular member having a proximal end and a distal end and defining a passage with a bend-enhancing region being arranged intermediate the proximal end and the distal end. The bend-enhancing region is constituted by an array of longitudinally spaced slots. Each of at least some of the slots is substantially L-shaped and comprises a first portion arranged transversely to a longitudinal axis of the tubular member and a second portion arranged at an angle to the first portion. An actuator cooperates with the tubular member to effect bending of the tubular member about the bend-enhancing region of the tubular member.

### CROSS-REFERENCE TO RELATED APPLICATIONS

This is a national phase entry under 35 U.S.C. §371 of International Patent Application PCT/AU2010/000022, filed Jan. 8, 2010, published in English as International Patent Publication WO 2010/081187 A1 on Jul. 22, 2010, which claims the benefit under Article 8 of the Patent Cooperation Treaty to U.S. Provisional Patent Application Ser. No. 61/145,036 filed on Jan. 15, 2009, the disclosure of each of which is hereby incorporated herein by this reference in its entirety.

### TECHNICAL FIELD

This disclosure relates, generally, to a catheter and, more particularly, to a steerable stylet for a catheter, to a component for a steerable stylet and to a catheter including the steerable stylet.

### BACKGROUND

Electrophysiology catheters are medical devices used for measuring electrical signals within the heart and are often used in the diagnosis of various arrhythmias. Certain types of these catheters may also be used for treating arrhythmias through ablative techniques.

Generally, to access the region of the heart to be treated, the catheter is inserted through the femoral vein of the patient. The tip of the catheter is steered through the vascular system of the patient to the desired location. Similarly, the catheter tip is steered through the ventricles of the heart to be positioned at the desired location.

Steerable catheters have, in the past, made use of a metal strip or shim contained within the distal end of the catheter as a portion of a steering device, which generally includes a tube with slots arranged transversely to a longitudinal axis of the tube. One or more pull wires are connected to the metal strip. Manipulation of these pull wires causes the metal strip to bend to deflect the distal end of the catheter. The junction between the slots and the remainder of the tube results in a small hinge area, which is prone to breakage. When breakage occurs, steerability or shape-forming ability is lost.

In addition, bending is generally only able to occur in one direction and to achieve bending in another direction, the entire catheter assembly must be rotated through the required angle before bending in the other direction is able to be effected.

### SUMMARY

According to a first aspect, there is provided a steerable stylet that includes an elongate tubular member having a proximal end and a distal end and defining a passage with a bend-enhancing region being arranged intermediate the proximal end and the distal end, the bend-enhancing region being constituted by an array of longitudinally spaced slots, each of at least some of the slots being substantially L-shaped and comprising a first portion arranged transversely to a longitudinal axis of the tubular member and a second portion arranged at an angle to the first portion; and an actuator that cooperates with the tubular member to effect bending of the tubular member about the bend-enhancing region of the tubular member.

Opposed walls of the first portion of each of the at least some of the slots may diverge from a junction with the second portion to a surface of the tubular member.

In an embodiment, a medial plane bisecting the first portion of each of the at least some of the slots may be arranged at an acute angle to a plane extending normal to the longitudinal axis of the tubular member. The first portion may be arranged at an obtuse angle relative to its associated second portion, the second portion extending towards a proximal end of the tubular member and being arranged at an acute angle relative to a plane parallel to the longitudinal axis of the tubular member.

In another embodiment, the first portion of each of the at least some of the slots may be arranged substantially normal to a plane parallel to the longitudinal axis of the tubular member. In this embodiment, opposed walls of the first portion may be substantially parallel to each other. Also, in this embodiment, the first portion may be arranged at an acute angle relative to its associated second portion.

In the case of either of the embodiments described above, a proximal end of the second portion may be shaped to relieve stress. Thus, the proximal end of the second portion may have an enlarged rounded shape to provide stress relief to inhibit breakage at the junction between the proximal end of the second portion and the tubular member. However, even if breakage does occur, the segments resulting from the breakage are constrained against rotation relative to each other because of the shape of the slots and because the actuator is a close fit in the lumen of the tubular member.

In an embodiment, the slots may be arranged in groups. The spacing between the slots of one group may differ with respect to the spacing between the slots of at least one other group.

In this embodiment, the stylet may include a control member displaceably arranged relative to the tubular member, the control member interacting with the bend-enhancing region of the tubular member for controlling the extent of deflection of a distal part of the tubular member.

By “the extent of deflection of the distal part of the tubular member” is generally meant the size of the radius of curvature of the deflected distal part of the tubular member. However, the tubular member may be able to be deflected in ways other than into a curved shape, for example, into a helical shape, and the control member may be operable to control the shape of such helical deflection. The terminology “the extent of deflection of the distal part of the tubular member” is intended to cover all such applications.

The control member may be configured to impede the bend-enhancing region of the tubular member for controlling the extent of deflection of the distal part of the tubular member. Thus, for example, the control member may be a sleeve received in or over the tubular member, the sleeve being axially displaceable relative to the tubular member.

The slots may divide the elongate tubular member into segments, adjacent segments having cooperating torque transmission elements for assisting in torque transmission during rotation of the tubular member in use. The cooperating torque transmission elements may comprise complementary engaging formations. A first engaging formation may comprise a tab centrally arranged and protruding longitudinally from one segment. A second engaging formation may comprise a slot, having a shape that is complementary to that of the tab, defined in the adjacent segment with the tab being received with limited, if any, clearance in the slot.

In all embodiments, each of the slots in the array of slots may have the L-shape, i.e., each slot may comprise the first portion arranged transversely to the longitudinal axis of the tubular member and the second portion arranged at an angle to the first portion.

The actuator may be received in the passage of the tubular member, with the actuator being fast with the tubular member distally of the bend-enhancing region to effect bending of the tubular member about the bend-enhancing region by relative longitudinal manipulation between the tubular member and the actuator. One of the tubular member and the actuator may extend distally beyond the point where the tubular member and the actuator are made fast with each other. The distally extending part of the tubular member and/or the actuator, as the case may be, may be bent into a desired shape or may be capable of being bent into a desired shape, such as a loop shape.

The actuator may be a wire received in the passage of the tubular member. Instead, the actuator may, itself, be tubular.

According to a second aspect, there is provided a component for a steerable stylet, the component including:
an elongate tubular member having a proximal end and a distal end and defining a passage with a bend-enhancing region being arranged intermediate the proximal end and the distal end; and
an array of longitudinally spaced slots constituting the bend-enhancing region, each of at least some of the slots being substantially L-shaped and comprising a first portion arranged transversely to a longitudinal axis of the tubular member and a second portion arranged at an angle to the first portion.

According to a third aspect, there is provided a catheter that includes:
an electrode sheath defining a lumen; and
a steerable stylet, as described above, received in the lumen of the electrode sheath.

### BRIEF DESCRIPTION OF THE DRAWINGS

FIG. 1 shows a three-dimensional view of a distal part of a first embodiment of a steerable stylet;

FIG. 2 shows a side view of the distal part of the steerable stylet of FIG. 1;

FIG. 3 shows a side view of a distal part of a second embodiment of a steerable stylet;

FIG. 4 shows a plan view of the steerable stylet of FIG. 3;

FIG. 5 shows a side view of the distal part of the steerable stylet of FIGS. 1 and 2 with a break in a component of the stylet;

FIG. 6 shows a three-dimensional view of the distal part of the steerable stylet of FIGS. 1 and 2 with the parts of the component separated at the break;

FIG. 7 shows a three-dimensional view of a catheter including the stylet of FIGS. 1 and 2;

FIG. 8 shows a three-dimensional view of a catheter including the stylet of FIGS. 3 and 4;

FIG. 9 shows a three-dimensional view of a distal part of a third embodiment of a steerable stylet;

FIG. 10 shows a side view of a distal part of a fourth embodiment of a steerable stylet;

FIG. 11 shows, on an enlarged scale, a side view of the part of the stylet of FIG. 10 labeled “X”;

FIG. 12 shows a schematic plan view of a part of a tubular member of the stylet of FIG. 10 laid flat; and

FIG. 13 shows, on an enlarged scale, the part of the tubular member of FIG. 12 labeled “Y.”

### DETAILED DESCRIPTION OF THE INVENTION

In the drawings, reference numeral 10 generally designates an embodiment of a steerable stylet. The stylet 10 comprises an elongate, tubular member 12 defining a passage 14. The tubular member 12 has a proximal end (not shown) and a distal end 12.1. The stylet further comprises an actuator 16, which, in the illustrated embodiment, is received in the passage 14 of the tubular member 12.

The tubular member 12 defines a bend-enhancing region 18 arranged proximally of the distal end 12.1 of the tubular member 12. The bend-enhancing region 18 is constituted by a plurality of longitudinally spaced slots 20. In the illustrated embodiment, each slot 20 of the bend-enhancing region 18 is cranked, or substantially L-shaped. Thus, each slot 20 includes a first portion 22 arranged transversely to a longitudinal axis of the tubular member 12 and a second portion 24 arranged at an angle relative to the first portion 22. The second portion 24 extends toward the proximal end of the tubular member 12.

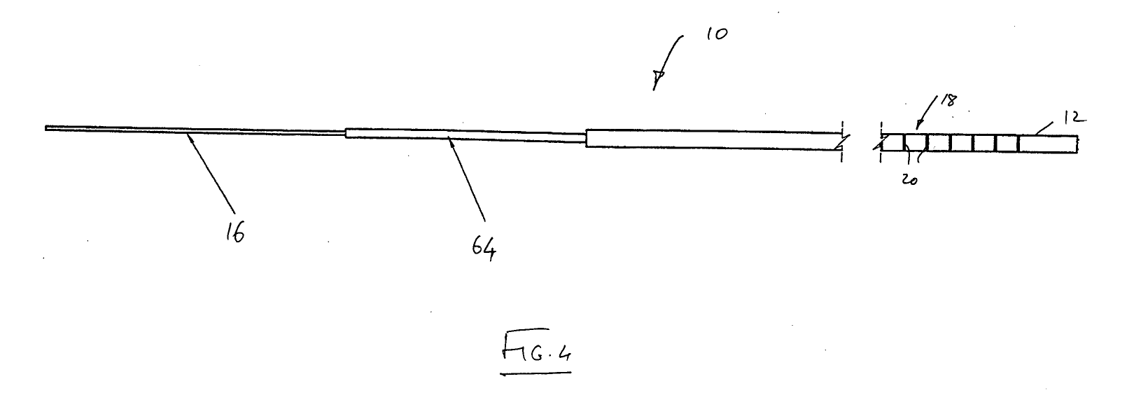

The first portion 22 of each slot 20 comprises a pair of opposed walls 26, which diverge from a junction 28 with the second portion 24 toward a surface 30 of the tubular member 12. A medial plane, illustrated schematically at 32 in FIG. 2 of the drawings, bisecting the first portion 22 of the slot 20 is arranged at a predetermined angle “A” relative to a plane 34 arranged orthogonally to the longitudinal axis of the tubular member 12. Typically, the angle “A” is of the order of approximately 0°-45°, more particularly, about 5°-35° and, preferably, about 10°-30°.

An included angle “B” between the first portion 22 and the second portion 24 is an obtuse angle. In this embodiment, the second portion 24 extends substantially parallel to the longitudinal axis of the tubular member 12 or, instead, is inclined slightly upwardly towards a proximal end of the second portion 24, such that the obtuse angle “B” is of the order of 90°-135° and, more particularly, about 100°-125° and, preferably, about 110°-120°.

A distal end 16.1 of the actuator 16 is secured to the distal end 12.1 of the tubular member 12. Hence, relative longitudinal displacement between the tubular member 12 and the actuator 16 results in bending of the stylet 10 at the bend-enhancing region 18 to allow deflection or steering of the distal part of the stylet 10.

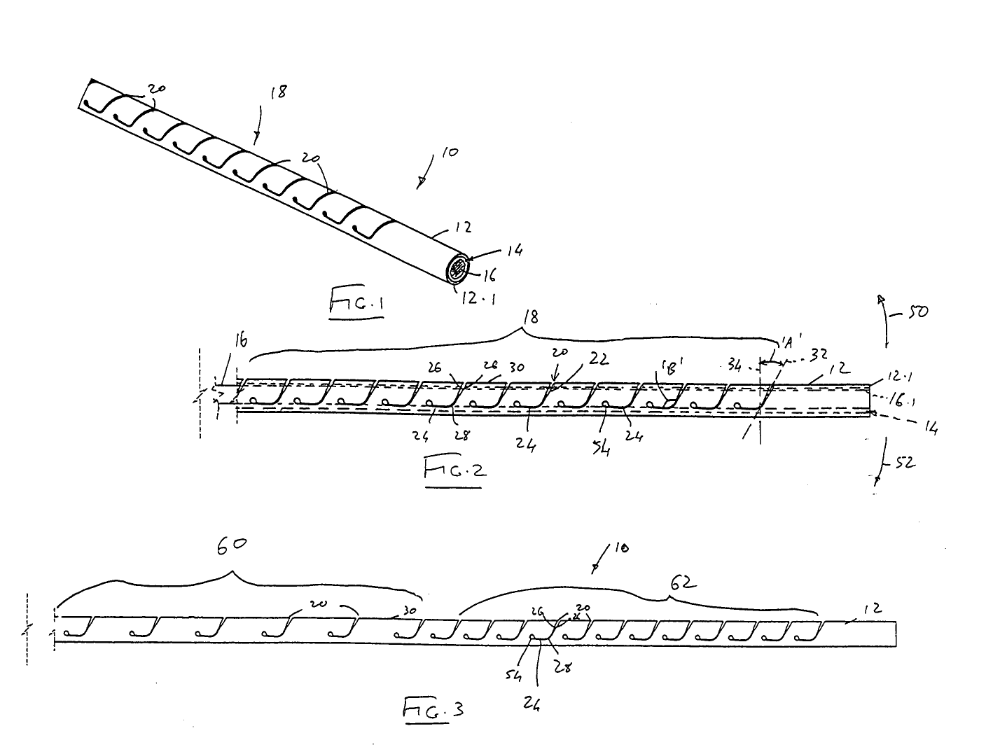

In the embodiment illustrated in FIGS. 1 and 2 of the drawings, the actuator 16, which is in the form of a metal wire, is secured to a mounting member 36 (FIG. 7), which, in turn, is attached via a bayonet fitting 38 to a proximal end of a handle 40 of a catheter 42. The tubular member 12 is attached to a mounting element (not shown), which is slidably arranged relative to the mounting member 36 and which is attached to a steering control member 44 slidably arranged on a distal part of the handle 40 of the catheter 42. By relative movement between the tubular member 12 and the actuator 16, bending of the stylet 10 and, hence, an electrode sheath 46 in which the stylet 10 is received, is effected to cause displacement of a distal region of the electrode sheath 46 from the position shown in solid lines in FIG. 7 to the position shown in dotted lines and vice versa. It is to be noted that the distal end of the electrode sheath 46 has a plurality of electrodes 48.

The tubular member 12 is made of any suitable resilient and flexible material and may be made of a suitable metal such as stainless steel, a shape-memory alloy such as a nickel-titanium alloy, a suitable plastics material such as polyetheretherketone (PEEK), or the like.

In use, when it is desired to bend the stylet 10 in the direction of arrow 50 (FIG. 2), the steering control member 44 of the catheter 42 is operated to cause relative displacement between the tubular member 12 and the actuator 16, bending the tubular member 12 in this direction by causing closing of the wedge shape of the portion 22 of each of the slots 20.

An advantage of this embodiment is that bending can also be effected in the direction of arrow 52 by appropriate manipulation of the steering control member 44 of the catheter 40. When the tubular member 12 is bent in the direction of arrow 52, the portions 24 of the slots 20 open to create a long hinge region and, therefore, low stresses allowing bending in the direction of the arrow 52.

In this regard, it is to be noted that a proximal part 54 of the portion 24 of each slot 20 is shaped to relieve stress. More particularly, the proximal end of each portion 24 is a rounded, enlarged, bulbous shape to provide stress relief. This lowers the probability of breakage at the hinging region of each slot 20.

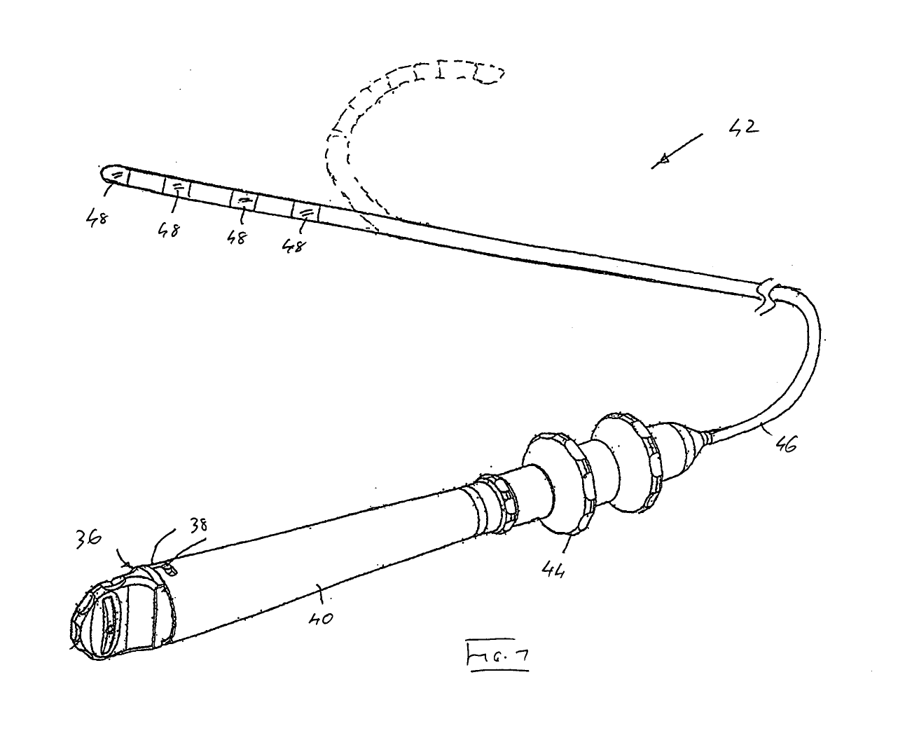

However, if such a breakage does occur such as, for example, as shown at 56 in FIGS. 5 and 6 of the drawings, the shapes of the slots 20 means that segments 58 of the tubular member 12 resulting from the break remain interlocked. Thus, the segments 58 are inhibited from rotating relative to each other and the stylet 10 can still be used in a normal way to steer the electrode sheath 46 of the catheter 42. In this regard, it is to be noted that, because the actuator 16 extends through the passage 14 of the tubular member 12, the actuator 16 assists in retaining the segments 58 in position relative to each other and inhibiting separation of the segments 58. It will also be understood that the lumen of the catheter sheath 46 of the catheter 42 constrains the segments 58 against separation.

Under normal operating conditions of the stylet 10, i.e., without breakage, the long hinge regions provided by the portions 24 of the slots 20 have low stresses and allow the tubular member 12 to twist with minimal likelihood of breakage. In addition, because the shapes of segments of the tubular member defined between adjacent slots 20 effectively cause interlocking, torque transfer along the tubular member 12 is facilitated.

In FIGS. 3, 4 and 8 of the drawings, another embodiment of the stylet is illustrated. With reference to the previous embodiment, like reference numerals refer to like parts, unless otherwise specified.

In this embodiment, the slots 20 are arranged in two discrete groups 60, 62. While two such groups are illustrated, it will be appreciated that the slots 20 could be arranged in a greater or a fewer number of groups as desired.

The group 62 has the slots 20 spaced more closely than the slots 20 of the group 60. Hence, a tighter radius can be effected using the slots 20 of the group 62 than the slots 20 of the group 60.

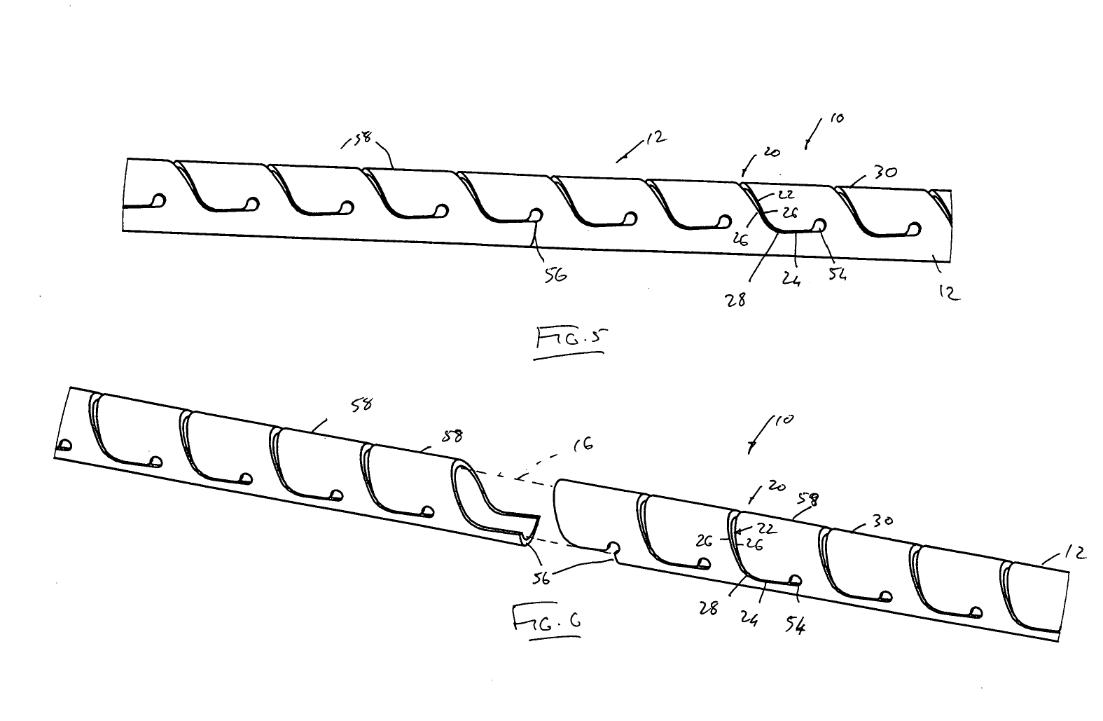

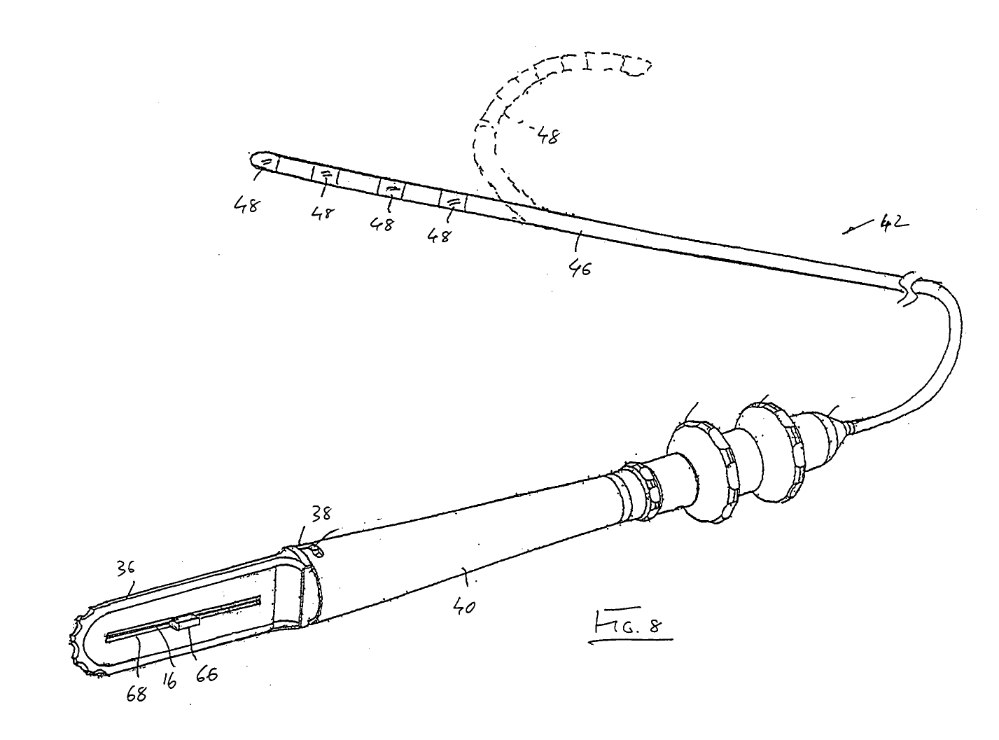

In this embodiment, a control member in the form of a tube 64 (FIG. 4) is received over the actuator 16 within the passage 14 of the tubular member 12. The tube 64 is connected to a slide 66 (FIG. 8) carried by the mounting member 36 of the catheter 40. It is to be noted in this embodiment that the mounting member 36 is an elongate member defining a slot 68 in which the slide 66 is displaceable. The actuator 16 extends through the slot 68 and is fast with a proximal end of the mounting member 36. The tube 64 can, therefore, slide relative to the tubular member and can block or occlude a predetermined number of slots 20 as desired. Thus, with this arrangement, either the slots 20 of the group 62 can be used where a tighter radius of curvature is required or the slots 20 of the group 60 can be used where a larger radius of curvature is required by appropriate manipulation of the tube 64.

Therefore, by varying the position of the tube 64 relative to the tubular member 12, a variable deflection of the distal part of the tube 12 can be achieved and, consequently, a distal part of the electrode sheath 46. This creates greater flexibility and versatility enabling a clinician more accurately to position a distal part of the catheter sheath 46 of the catheter 42.

Referring to FIG. 9 of the drawings, a third embodiment of a steerable stylet 10 is illustrated. Once again, with reference to the previous embodiments, like reference numerals refer to like parts unless otherwise specified.

In this embodiment, a distal part 70 of the actuator 16 protrudes beyond the part 16.1 of the actuator, which is secured to the distal end 12.1 of the tubular member 12 of the stylet 10. The part 70 is bent into a predetermined shape, which, in this case, is a loop shape 72. The part 70 is cranked as shown at 74 so that the loop shape 72 lies in a plane transverse to the longitudinal axis of the tubular member 12.

The actuator 16 could be of a shape-memory alloy to be pre-formed with the loop shape 72 or, instead, the actuator 16 could be of a material that is able to be bent into the desired shape prior to use.

While the embodiment has been described with the actuator 16 extending beyond the tubular member 12, the same result could be achieved by having the tubular member 12 extend beyond the actuator 16. Further, both the tubular member 12 and the actuator 16 could have distal portions formed into the desired shape.

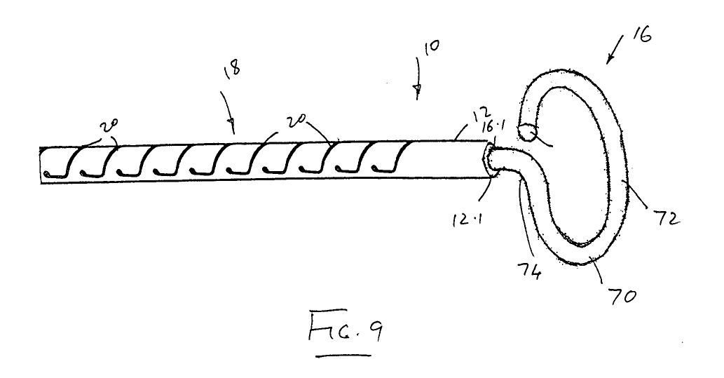

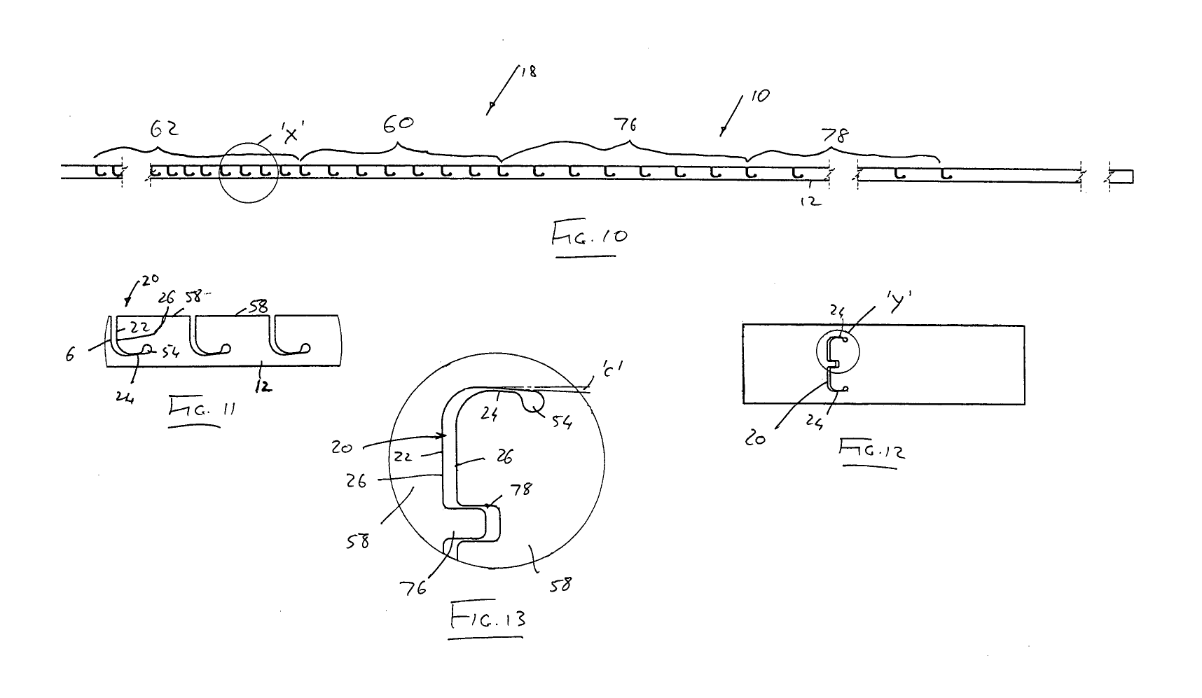

Referring now to FIGS. 10-13 of the drawings, yet a further embodiment of a steerable stylet 10 is illustrated. Once again, with reference to the previous embodiments, like reference numerals refer to like parts, unless otherwise specified.

In this embodiment, the slots 20 are, once again, arranged in groups. There are four groups 60, 62, 76 and 78 of slots 20 where the pitch or spacing between the slots 20 of the groups differ with the space between the slots 20 of the group 62 being less than the spacing between the slots 20 of the group 60. The spacing between the slots 20 of the group 76 is greater than the spacing between the slots 20 of the group 60 and, similarly, the spacing between the slots 20 of the group 78 is greater than the spacing between the slots 20 of the group 76. By manipulation of the control member 64, the appropriate group or groups of slots 20 can be blocked by the control member 64 to vary the deflection of the distal part of the tubular member 12 of the stylet 10.

In this embodiment, the first portion 22 of each slot 20 is, as shown in greater detail in FIG. 11 of the drawings, substantially normal to the longitudinal axis of the tubular member 12. It is also to be noted that the opposed walls 26 of the first portion 22 of each slot are substantially parallel to each other and do not diverge.

The second portion 24 of each slot 20 projects at an acute angle “C” (FIG. 13) to a plane lying parallel to the longitudinal axis of the tubular member 12. The angle “C” is, typically, of the order of 1°-10°, more particularly, about 2°-5° and, preferably, about 3°. By having the second portion 24 of each slot 20 angled in this manner, improved interlocking between adjacent segments 58 of the tubular member 12 occurs. This means that if a breakage does occur between adjacent segments 58 of the tubular member 12, relative rotation between the segments 58 is inhibited. Thus, the stylet 10 can still be operated in its normal condition, as described above.

Further, to provide improved torque transmission, adjacent segments 58 of the tubular member 12 have complementary torque transmission elements for assisting in torque transmission during rotation of the tubular member 12 about its longitudinal axis. The torque transmission elements 12 comprise a tongue or tab 76 and a complementary groove 78 carried on adjacent segments 58. The tab 76 carried on one segment 58 is received with little, if any, clearance in its complementary groove 78 defined in the adjacent segment 58 as shown more clearly in FIG. 13 of the drawings. It is to be noted that the tab 76 projects toward the proximal end of the tubular member 12. Further, the tab 76 is located substantially equidistantly between opposed second portions 24 of the relevant slot 20. The complementary tab 76 and slot 78 further serve to locate adjacent segments 58 with respect to each other should breakage of the tubular member occur between those adjacent segments 58.

It is, therefore, an advantage of the described embodiments that bi-directional bending of the stylet 10 can be achieved. Due to the shape of the slots 20 and the resulting interlocking of the segments of the tubular member 12, torque transmission is facilitated. Also, should a breakage occur, the stylet 10 is still able to function substantially normally.

In addition, the stylet 10 can be configured to provide a variable deflection. Also, by making the stylet 10 of a low-cost material, it can be implemented as a one off, disposable, low-cost item.

It will be appreciated by persons skilled in the art that numerous variations and/or modifications may be made to the described embodiments without departing from the scope of the disclosure as broadly described. The present embodiments are, therefore, to be considered in all respects as illustrative and not restrictive.

1. A steerable stylet that includes:
an elongate tubular member having a proximal end and a distal end and defining a passage with a bend-enhancing region being arranged intermediate the proximal end and the distal end, the bend-enhancing region being constituted by an array of longitudinally spaced slots, each of at least some of the slots being substantially L-shaped and comprising a first portion arranged transversely to a longitudinal axis of the tubular member and a second portion arranged at an angle to the first portion; and
an actuator that cooperates with the tubular member to effect bending of the tubular member about the bend-enhancing region of the tubular member.

2. The stylet of claim 1 in which opposed walls of the first portion of each of the at least some of the slots diverge from a junction with the second portion to a surface of the tubular member.

3. The stylet of claim 1 in which a medial plane bisecting the first portion of each of the at least some of the slots is arranged at an acute angle to a plane extending normal to the longitudinal axis of the tubular member.

4. The stylet of claim 1 in which the first portion is arranged at an obtuse angle relative to its associated second portion, the second portion extending towards a proximal end of the tubular member and being arranged at an acute angle relative to a plane parallel to the longitudinal axis of the tubular member.

5. The stylet of claim 1 in which the first portion of each of the at least some of the slots is arranged substantially normal to a plane parallel to the longitudinal axis of the tubular member.

6. The stylet of claim 5 in which the first portion is arranged at an acute angle relative to its associated second portion.

7. The stylet of claim 1 in which a proximal end of each second portion is shaped to relieve stress.

8. The stylet of claim 1 in which the slots are arranged in groups.

9. The stylet of claim 8 in which the spacing between the slots of one group differs with respect to the spacing between the slots of at least one other group.

10. The stylet of claim 9, which includes a control member displaceably arranged relative to the tubular member, the control member interacting with the bend-enhancing region of the tubular member for controlling the extent of deflection of a distal part of the tubular member.

11. The stylet of claim 1 in which the slots divide the elongate tubular member into segments, adjacent segments having cooperating torque transmission elements for assisting in torque transmission during rotation of the tubular member in use.

12. The stylet of claim 1 in which each of the slots in the array of slots comprises the first portion arranged transversely to the longitudinal axis of the tubular member and the second portion arranged at an angle to the first portion.

13. The stylet of claim 1 in which the actuator is received in the passage of the tubular member, a distal end of the actuator being fast with the tubular member distally of the bend-enhancing region to effect bending of the tubular member about the bend-enhancing region by relative longitudinal manipulation between the tubular member and the actuator.

14. The stylet of claim 1 in which the actuator is a wire received in the passage of the tubular member.

15. A component for a steerable stylet, the component including:
an elongate tubular member having a proximal end and a distal end and defining a passage with a bend-enhancing region being arranged intermediate the proximal end and the distal end; and
an array of longitudinally spaced slots constituting the bend-enhancing region, each of at least some of the slots being substantially L-shaped and comprising a first portion arranged transversely to a longitudinal axis of the tubular member and a second portion arranged at an angle to the first portion.

16. A catheter that includes:
an electrode sheath defining a lumen; and
a steerable stylet, as claimed in claim 1, received in the lumen of the electrode sheath.
